(19) United States
(12) Patent Application Publication
(10) Pub. No.: US 2026/0076636 A1
(43) Pub. Date: Mar. 19, 2026

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Taiga GOTO, Tokyo (JP)

(73) Assignee: FUJIFILM Corporation, Tokyo (JP)

(21) Appl. No.: 19/321,328

(22) Filed: Sep. 8, 2025

(30) Foreign Application Priority Data
Sep. 19, 2024 (JP) 2024-162545

(51) Int. Cl. A61B 6/00; A61B 6/03; G06T 11/00

(52) U.S. Cl. CPC A61B 6/5258; G06T 12/30; A61B 6/032; G06T 2211/448

(57) ABSTRACT

An information processing apparatus includes a processor, in which the processor acquires first projection data output from a detector that has detected radiation of a first energy transmitted through a subject and second projection data output from the detector that has detected radiation of a second energy transmitted through the subject, the second energy being different from the first energy, and performs a correction process of correcting artifacts in a region in which an amount of change between the first projection data and the second projection data is equal to or greater than a threshold value.

### CROSS-REFERENCE TO RELATED APPLICATION

This application claims priority under 35 USC 119 from Japanese Patent Application No. 2024-162545 filed on Sep. 19, 2024, the disclosure of which is incorporated by reference herein.

### BACKGROUND

### 1. Technical Field

The present disclosure relates to an information processing apparatus, an information processing method, and an information processing program.

### 2. Description of the Related Art

A medical image is captured by irradiating a subject with radiation using a computed tomography (CT) apparatus or the like. For example, in a case where metal is included in an imaging target, such as in a case where a bolt used for bone fixation is present in a subject, the captured medical image may include artifacts caused by an influence of the metal.

Therefore, a technology for removing artifacts from the medical image is known. For example, in technologies described in JP2017-131307A and JP2019-122553A, a metal region is specified from a CT image generated by reconstructing projection data, and projection data correction is performed using projection data of the metal region obtained by forward-projecting a metal region image, thereby removing an influence of artifacts.

### SUMMARY

In the technologies described in JP2017-131307A and JP2019-122553A, the metal region is specified from the CT image.

Since this CT image includes artifacts, an error may occur in a case where the metal region is specified. In a case where the metal region image including an error is forward-projected in this way, an error occurs in forward-projection data, and forward-projection of the metal region image causes blurring due to an interpolation process in the forward-projection process. Therefore, there have been cases where the projection data cannot be appropriately corrected.

The present disclosure has been made in consideration of the above-described circumstances, and an object of the present disclosure is to provide an information processing apparatus, an information processing method, and an information processing program capable of improving accuracy of artifact correction.

In order to achieve the above-described object, according to a first aspect of the present disclosure, there is provided an information processing apparatus comprising: a processor, in which the processor is configured to: acquire first projection data output from a detector that has detected radiation of a first energy transmitted through a subject and second projection data output from the detector that has detected radiation of a second energy transmitted through the subject, the second energy being different from the first energy; and perform a correction process of correcting artifacts in a region in which an amount of change between the first projection data and the second projection data is equal to or greater than a threshold value.

According to a second aspect, in the information processing apparatus of the first aspect, the processor is configured to: perform, as the correction process, a process of correcting the region of correction target data by using interpolation data, the correction target data being the first projection data and the second projection data, or difference data between the first projection data and the second projection data; and generate an interpolation-processed reconstructed image by reconstructing the corrected correction target data.

According to a third aspect, in the information processing apparatus of the second aspect, the processor is configured to: generate an interpolation error reduction image in which an interpolation error component is reduced from the interpolation-processed reconstructed image; generate interpolation error reduction forward-projection data obtained by forward-projecting the interpolation error reduction image; perform a replacement process on the region of the correction target data based on the interpolation error reduction forward-projection data such that continuity between the region and an adjacent region is increased; and perform a residual error reduction process on the correction target data after the replacement to generate corrected projection data.

According to a fourth aspect, in the information processing apparatus of the third aspect, the processor is configured to: perform the replacement process by using any of baseline shift or normalized interpolation.

According to a fifth aspect, in the information processing apparatus of the third aspect, the processor is configured to: perform the residual error reduction process on error projection data obtained by subtracting a metal component corresponding to metal and the interpolation error reduction forward-projection data from the correction target data.

According to a sixth aspect, in the information processing apparatus of the third aspect, the processor is configured to: perform the residual error reduction process based on frequency information.

According to a seventh aspect, in the information processing apparatus of the sixth aspect, the processor is configured to: perform, as the residual error reduction process, a weighted addition in which a weight of a frequency component other than a high-frequency component corresponding to noise and a low-frequency component corresponding to artifacts is greater than weights of the high-frequency component and the low-frequency component.

According to an eighth aspect, in the information processing apparatus of the third aspect, the processor is configured to: generate an interpolation error reduction image in which a pixel value of a pixel having a pixel value within a predetermined range is replaced with a pixel value different from the pixel value.

According to a ninth aspect, in the information processing apparatus of the first aspect, the processor is configured to: derive the amount of change based on a difference or a ratio between the first projection data and the second projection data.

In order to achieve the above-described object, according to a tenth aspect of the present disclosure, there is provided an information processing method comprising: causing a processor to execute: acquiring first projection data output from a detector that has detected radiation of a first energy transmitted through a subject and second projection data output from the detector that has detected radiation of a second energy transmitted through the subject, the second energy being different from the first energy; and performing a correction process of correcting artifacts in a region in which an amount of change between the first projection data and the second projection data is equal to or greater than a threshold value.

In order to achieve the above-described object, according to an eleventh aspect of the present disclosure, there is provided an information processing program for causing a processor to execute a process comprising: acquiring first projection data output from a detector that has detected radiation of a first energy transmitted through a subject and second projection data output from the detector that has detected radiation of a second energy transmitted through the subject, the second energy being different from the first energy; and performing a correction process of correcting artifacts in a region in which an amount of change between the first projection data and the second projection data is equal to or greater than a threshold value.

According to the present disclosure, it is possible to improve accuracy of artifact correction.

### BRIEF DESCRIPTION OF THE DRAWINGS

FIG. 1 is a configuration diagram showing an example of a configuration of a CT apparatus of an embodiment.

FIG. 2 is a configuration diagram showing an example of a configuration of a console of the embodiment.

FIG. 3 is a functional block diagram showing an example of a function of the console of the embodiment.

FIG. 4 is a flowchart showing an example of a flow of information processing of the embodiment.

### DESCRIPTION OF THE PREFERRED EMBODIMENTS

Embodiments of the present invention will be described in detail below with reference to the drawings. It should be noted that the present embodiment is not intended to limit the present invention.

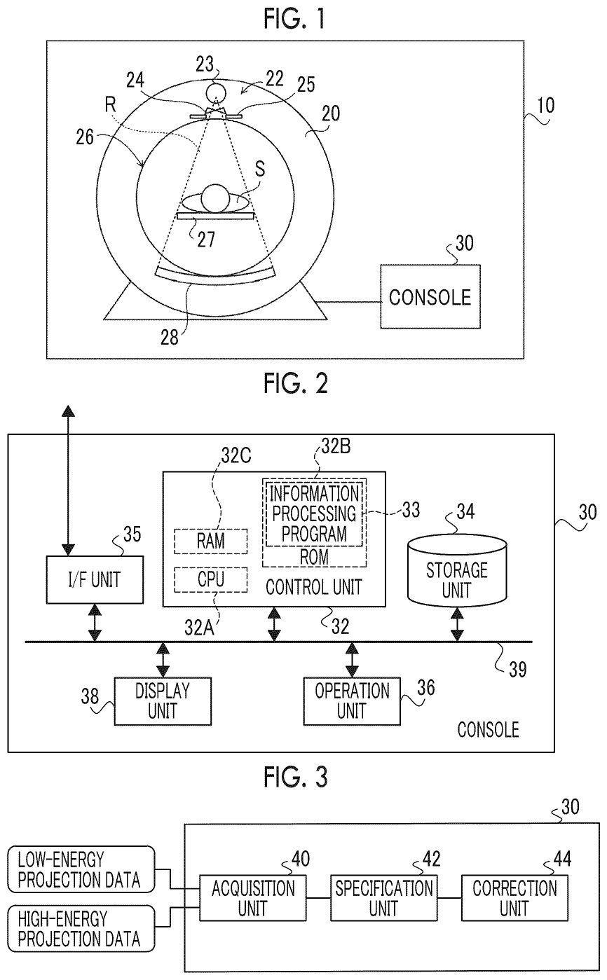

First, an example of a configuration of a computed tomography (CT) apparatus of the present embodiment will be described. In FIG. 1, a configuration diagram showing an example of a configuration of a CT apparatus 10 of the present embodiment is shown. As shown in FIG. 1, the CT apparatus 10 of the present embodiment comprises a gantry 20, a patient table 27, and a console 30.

The gantry 20 includes an opening portion 26, and a subject S as an imaging target is disposed in the opening portion 26 in a state of being placed on the patient table 27. The gantry 20 and the patient table 27 are configured to move relative to each other in a direction passing through the opening portion 26.

Inside the gantry 20, a radiation generation device 23 including a radiation tube (not shown), a bowtie filter 24, and a collimator 25, and a detector 28 are disposed in a state of facing each other with the subject S interposed therebetween. Radiation R emitted from the radiation generation device 23 is shaped into a beam shape suitable for a size of the subject S by the bowtie filter 24 and the collimator 25 and is emitted to the subject S. The detector 28 detects the radiation transmitted through the subject S and generates a projection image corresponding to a dose of the detected radiation. The detector 28 of the present embodiment is a photon-counting type detector in which a plurality of detection elements (not shown) that detect photon energy, which is the energy of photons of incident radiation, are disposed in an arc shape centered on a focal point of the radiation tube of the radiation generation device 23. The detector 28, which is a photon-counting type detector, outputs a projection image corresponding to the photon energy.

The radiation generation device 23 and the detector 28 are rotated around the subject S by a rotation drive unit (not shown) of the gantry 20. The radiation irradiation from the radiation generation device 23 and the radiation detection by the detector 28 are repeated while both the radiation generation device 23 and the detector 28 are rotated, thereby acquiring projection data at various projection angles. A plurality of pieces of projection data detected by the detector 28 are output to the console 30.

The console 30 of the present embodiment performs various controls related to imaging, generation of a medical image, and the like. The medical image generated by the console 30 is output to an external device (not shown) such as a picture archiving and communication system (PACS) via a network.

The console 30 of the present embodiment is an example of an information processing apparatus of the present disclosure. As an example, the console 30 of the present embodiment is a server computer. The console 30 comprises, as shown in FIG. 2, a control unit 32, a storage unit 34, an interface (I/F) unit 35, an operation unit 36, and a display unit 38. The control unit 32, the storage unit 34, the I/F unit 35, the operation unit 36, and the display unit 38 are connected to each other via a bus 39, such as a system bus or a control bus, so as to be capable of transmitting and receiving various kinds of information.

The control unit 32 of the present embodiment controls the overall operation of the console 30. The control unit 32 comprises a central processing unit (CPU) 32A, a read only memory (ROM) 32B, and a random access memory (RAM) 32C. The ROM 32B stores, in advance, various programs including an information processing program 33, which will be described below, to be executed by the CPU 32A, and the like. The RAM 32C temporarily stores various kinds of data.

The storage unit 34 stores the projection data output from the detector 28, various other kinds of information, and the like. As specific examples, the storage unit 34 is implemented by a storage medium such as a hard disk drive (HDD), a solid state drive (SSD), and a flash memory.

The I/F unit 35 performs communication of various kinds of information with the rotation drive unit (not shown) of the gantry 20, the radiation generation device 23, and the detector 28 through wired communication or wireless communication. The console 30 of the present embodiment receives the projection data from the detector 28 via the I/F unit 35. The received projection data is stored in the storage unit 34.

The console 30 acquires the plurality of pieces of projection data from the detector 28 via the I/F unit 35. The control unit 32 performs a reconstruction process on the acquired plurality of pieces of projection data to generate a tomographic image which is a reconstructed image of the subject S.

The operation unit 36 is used by a user to input various kinds of information such as instructions related to image generation such as scan conditions for acquiring projection data and parameter instructions, and instructions related to image display. The operation unit 36 is not particularly limited, and examples of the operation unit 36 include various switches, buttons, a touch panel, a touch pen, a keyboard, and a mouse. The display unit 38 displays various kinds of information, a medical image, and the like. It should be noted that the operation unit 36 and the display unit 38 may be integrated into a touch panel display. Additionally, for example, the operation unit 36 may receive a voice input from the user.

The CT apparatus 10 of the present embodiment is a multi-energy CT. In the multi-energy CT, a plurality of types of projection data can be acquired by detecting, with the detector 28, radiation having a plurality of different energies. For example, in a case of dual-energy CT, which is a type of multi-energy CT, two types of projection data can be obtained: low-energy projection data corresponding to radiation of relatively low energy and high-energy projection data corresponding to radiation of relatively high energy. By using the obtained plurality of types of projection data, it is possible to generate, for example, a virtual monochromatic X-ray image.

An imaging method as the multi-energy CT by the CT apparatus 10 of the present embodiment, that is, a method for acquiring a plurality of types of projection data having different energies, is not particularly limited, and a known imaging method can be applied.

As an example, in the present embodiment, the CT apparatus 10 is a dual-energy CT, and at each of a plurality of projection angles, radiation of a first energy, which is relatively low, is emitted from the radiation generation device 23 to the subject S, and the low-energy projection data is acquired by the detector 28. In addition, radiation of a second energy, which is higher than the first energy, is emitted from the radiation generation device 23 to the subject S, and the high-energy projection data is acquired by the detector 28. Therefore, a plurality of pieces of low-energy projection data and a plurality of pieces of high-energy projection data are output from the detector 28 to the console 30. The low-energy projection data of the present embodiment is an example of one of the first projection data and the second projection data of the present disclosure, and the high-energy projection data of the present embodiment is an example of one of the first projection data and the second projection data of the present disclosure.

In FIG. 3, a functional block diagram showing an example of a function of the console 30 is shown. The console 30 comprises an acquisition unit 40, a specification unit 42, and a correction unit 44. As an example, the console 30 of the present embodiment executes the information processing program 33, so that the CPU 32A of the control unit 32 functions as the acquisition unit 40, the specification unit 42, and the correction unit 44.

The acquisition unit 40 has a function of acquiring the plurality of pieces of low-energy projection data and the plurality of pieces of high-energy projection data output from the detector 28. Specifically, the acquisition unit 40 acquires, via the I/F unit 35, from the detector 28, the low-energy projection data and the high-energy projection data, which are captured by radiation of two types of energies sequentially emitted in each of a plurality of directions with respect to the subject S, as mentioned above. It is preferable that both pieces of projection data are acquired at substantially the same position in order to observe a change between the low-energy projection data and the high-energy projection data. For example, imaging using a two-layer type detector 28, a dual-rotation method, photon counting computed tomography (PCCT), or the like is preferable. The acquisition unit 40 may acquire, from the storage unit 34, the low-energy projection data and the high-energy projection data that have been once acquired from the detector 28 and stored in the storage unit 34. The acquisition unit 40 outputs the acquired low-energy projection data and high-energy projection data to the specification unit 42.

The specification unit 42 has a function of specifying a predetermined region in a projection space. The specification unit 42 of the present embodiment specifies a metal region in each of the plurality of pieces of low-energy projection data and the plurality of pieces of high-energy projection data. The specification unit 42 specifies a region in which an amount of change between the low-energy projection data and the high-energy projection data is equal to or greater than a threshold value, as the metal region. The metal appearing in the low-energy projection data and the high-energy projection data together with the subject S exhibits a greater amount of change, as compared with a human tissue, in the low-energy projection data with respect to the high-energy projection data, or in the high-energy projection data with respect to the low-energy projection data. Therefore, a threshold value that allows distinction from the human tissue is obtained, and the specification unit 42 specifies a region in which the amount of change with respect to the high-energy projection data is equal to or greater than the threshold value, as the metal region. The specific threshold value need only be determined in advance according to the energy of the radiation to be emitted from the radiation generation device 23, the type of metal, and the like.

The specification unit 42 may specify the metal region based on an amount of change between pieces of spectral image data. For example, the acquisition unit 40 reconstructs a plurality of pieces of spectral projection data (correction target projection data) having different pieces of spectral information to generate a plurality of pieces of spectral image data having different pieces of spectral information. The specification unit 42 may specify a metal region in an image space based on the amount of change between the plurality of pieces of spectral image data and may specify the metal region in the high-energy projection data and the low-energy projection data by performing a forward-projection process on the specified metal region. Here, the spectral information includes any of photon energy, basis material information, effective atomic number information, electron density information, or scattered X-ray information.

The specification unit 42 outputs information indicating the metal region specified in each of the plurality of pieces of low-energy projection data and the plurality of pieces of high-energy projection data to the correction unit 44.

The correction unit 44 has a function of performing a correction process of correcting artifacts in the metal region specified by the specification unit 42. The correction process of correcting artifacts is a process of reducing an error caused by metal. The error caused by metal includes errors caused by beam hardening, noise, scattered rays, and the like.

Specifically, the correction unit 44 performs a process of correcting the metal regions in the low-energy projection data and the high-energy projection data with interpolation data in the projection space. Through the processing, low-energy interpolation-processed projection data corresponding to the low-energy projection data and high-energy interpolation-processed projection data corresponding to the high-energy projection data are generated. As the interpolation data, predetermined data can be applied according to the energy of the radiation to be emitted, the subject S, and the like. The interpolation data is data generated by an interpolation process. The interpolation process is a process of estimating projection data in a metal region, which is a metal region having a large error, from adjacent projection data having a small error. Specifically, examples of the interpolation process include a simple linear interpolation process from two adjacent pieces of projection data, and an interpolation process using as baseline shift and normalized interpolation by utilizing forward-projection data of an image in which artifacts are reduced. It should be noted that, here, the low-energy interpolation-processed projection data and the high-energy interpolation-processed projection data are collectively referred to simply as “interpolation-processed projection data”.

Additionally, the correction unit 44 reconstructs the interpolation-processed projection data to generate an interpolation-processed reconstructed image. Specifically, the correction unit 44 reconstructs the low-energy interpolation-processed projection data to generate a low-energy interpolation-processed reconstructed image, and reconstructs the high-energy interpolation-processed projection data to generate a high-energy interpolation-processed reconstructed image. It should be noted that, here, the low-energy interpolation-processed reconstructed image and the high-energy interpolation-processed reconstructed image are collectively referred to simply as an “interpolation-processed reconstructed image”.

In addition, the correction unit 44 performs an interpolation error reduction process on the interpolation-processed reconstructed image in order to remove residual errors (artifacts) remaining after the metal region has been replaced with interpolation data as described above, and generates an interpolation error reduction image. Specifically, the correction unit 44 performs the interpolation error reduction process on the low-energy interpolation-processed reconstructed image to generate a low-energy interpolation error reduction image, and performs the interpolation error reduction process on the high-energy interpolation-processed reconstructed image to generate a high-energy interpolation error reduction image. It should be noted that, here, the low-energy interpolation error reduction image and the high-energy interpolation error reduction image are collectively referred to simply as an “interpolation error reduction image”. The correction unit 44 of the present embodiment generates the interpolation error reduction image by replacing a pixel value of a pixel having a pixel value within a predetermined range with a pixel value different from the pixel value. Examples of such processing include a segmentation process. The predetermined range of pixel values can be defined in accordance with pixel values of residual errors (artifacts) remaining after the metal region has been replaced with the interpolation data. For example, the predetermined range may be defined based on a range of pixel values corresponding to a soft tissue, which is more susceptible to the influence of interpolation errors among bone tissues, soft tissues, and air tissues that constitute the human body. By replacing a pixel whose pixel value falls within the predetermined range with a representative CT value of the soft tissue, for example, with an average value of the pixel values within the predetermined range, the variation in CT values caused by interpolation errors can be suppressed. Instead of the average value, the replacement may be made with a predetermined pixel value according to the energy of the radiation to be emitted, the subject S, and the like.

Additionally, the correction unit 44 generates interpolation error reduction forward-projection data by forward-projecting the interpolation error reduction image into the projection space. Specifically, the correction unit 44 generates low-energy interpolation error reduction forward-projection data by forward-projecting the low-energy interpolation error reduction image into the projection space, and generates high-energy interpolation error reduction forward-projection data by forward-projecting the high-energy interpolation error reduction image into the projection space. It should be noted that, here, the low-energy interpolation error reduction forward-projection data and the high-energy interpolation error reduction forward-projection data are collectively referred to simply as, “interpolation error reduction forward-projection data”.

In addition, the correction unit 44 performs a residual error reduction process on the metal region in the low-energy projection data and the metal region in the high-energy projection data in order to reduce residual errors (artifacts) remaining even after the above-described processing, and generates corrected projection data. Specifically, the correction unit 44 performs a replacement process on the metal region in the low-energy projection data based on the interpolation error reduction forward-projection data such that continuity between the metal region and an adjacent region is increased. Further, the correction unit 44 performs the replacement process on the metal region in the high-energy projection data based on the interpolation error reduction forward-projection data such that continuity between the metal region and an adjacent region is increased. As the replacement process, for example, any of baseline shift or normalized interpolation may be used. Furthermore, the correction unit 44 performs the residual error reduction process on the low-energy projection data and high-energy projection data after the replacement, and generates corrected projection data. As an example, the correction unit 44 of the present embodiment performs the residual error reduction process on error projection data obtained by subtracting a metal component corresponding to metal and the interpolation error reduction forward-projection data from each of the low-energy projection data and the high-energy projection data.

Additionally, the correction unit 44 may perform the residual error reduction process based on frequency information. For example, the correction unit 44 removes a low-frequency component as noise and removes a high-frequency component that is higher than the reference, for example, a highest-frequency component, as photon noise. In the present embodiment, it is assumed that the error projection data is composed of noise, interpolation errors, metal artifacts, and structural components, and the highest-frequency component corresponding to a beam interval of the projection data is treated as noise, the low-frequency component is treated as metal artifacts, and the remaining components (hereinafter referred to as mid-frequency components) are treated as structural information. The correction unit 44 reduces the residual error by weighting and adding these components as the residual error reduction process. It should be noted that these weights may be set in advance according to the type of metal to be corrected, or the like, or empirically determined values verified in advance using various pieces of data may be used. Specifically, the separation into the high-frequency component, the low-frequency component, and the mid-frequency component may be performed by using a smoothing filter in a real space, or the separation may be performed by performing a filtering process in a frequency space after Fourier transform. The filtering process in the frequency space may also be incorporated as frequency modulation in the reconstruction filter. A weight of the mid-frequency component in the error projection data is set to be high, a weight of the low-frequency component is set to be low, and a weight of the high-frequency component is set to be low. The correction unit 44 performs the residual error reduction process by performing a weighted addition on the error projection data.

In addition, the correction unit 44 reconstructs the corrected projection data to generate a corrected reconstructed image. Specifically, the correction unit 44 reconstructs the low-energy corrected projection data to generate a low-energy corrected reconstructed image, and reconstructs the high-energy corrected projection data to generate a high-energy corrected reconstructed image. It should be noted that, here, the low-energy corrected reconstructed image and the high-energy corrected reconstructed image are collectively referred to simply as a “corrected reconstructed image”.

Next, an operation of the console 30 of the present embodiment will be described.

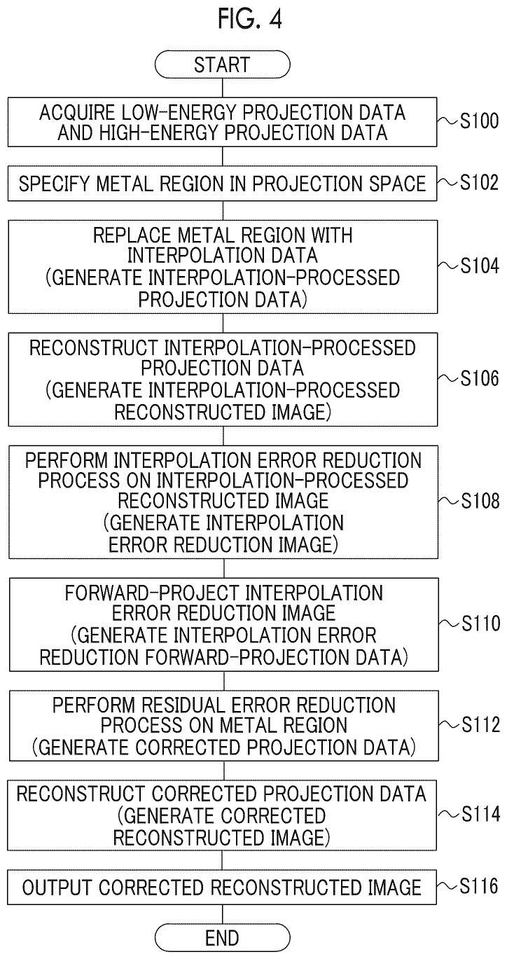

The console 30 of the present embodiment executes information processing shown as an example in FIG. 4 by the CPU 32A of the control unit 32 executing the information processing program 33 stored in the ROM 32B. In FIG. 4, a flowchart showing an example of a flow of the information processing by the console 30 of the present embodiment is shown.

First, in step S100 of FIG. 4, the acquisition unit 40 acquires the plurality of pieces of low-energy projection data and the plurality of pieces of high-energy projection data, as mentioned above.

In the next step S102, the specification unit 42 specifies the metal region in the projection space from each of the low-energy projection data and the high-energy projection data, as mentioned above.

In the next step S104, the correction unit 44 generates the interpolation-processed projection data by replacing the metal region with the interpolation data, as mentioned above.

In the next step S106, the correction unit 44 reconstructs the interpolation-processed projection data to generate the interpolation-processed reconstructed image, as mentioned above.

In the next step S108, the correction unit 44 performs the interpolation error reduction process on the interpolation-processed reconstructed image to generate the interpolation error reduction image, as mentioned above.

In the next step S110, the correction unit 44 generates the interpolation error reduction forward-projection data by forward-projecting the interpolation error reduction image into the projection space, as mentioned above.

In the next step S112, the correction unit 44 performs the residual error reduction process on the metal region to generate the corrected projection data, as mentioned above.

In the next step S114, the correction unit 44 reconstructs the corrected projection data to generate the corrected reconstructed image, as mentioned above.

In the next step S116, the correction unit 44 outputs the corrected reconstructed image generated in step S114 described above to a predetermined output destination. The output destination may be the storage unit 34 of the console 30 or may be a device outside the console 30. The corrected reconstructed image generated in this manner can be used to generate, for example, a virtual monochromatic X-ray image.

In a case where processing of step S116 ends, the information processing shown in FIG. 4 ends.

As described above, in the console 30 of each of the above-described embodiments, the CPU 32A functions as the acquisition unit 40 to acquire the low-energy projection data output from the detector 28 that has detected radiation of low energy transmitted through the subject S and the high-energy projection data output from the detector 28 that has detected radiation of high-energy transmitted through the subject S. Additionally, the CPU 32A functions as the specification unit 42 to specify a region in which the amount of change between the low-energy projection data and the high-energy projection data is equal to or greater than a threshold value, as the metal region. Further, the CPU 32A functions as the correction unit 44 to perform the correction process of correcting artifacts in the metal region.

As described above, in the console 30 of the above-described embodiment, the metal region is specified in the projection space from the low-energy projection data and the high-energy projection data, which are not affected by artifacts or are less affected by artifacts, as compared with a case where the metal region is specified from a reconstructed image. Therefore, with the console 30 of the above-described embodiment, the accuracy of specifying the metal region is improved, thereby making it possible to improve the accuracy of artifact correction. In addition, with the console 30 of the above-described embodiment, it is possible to restore a structure that may be lost during artifact removal.

In the above-described embodiment, a form has been described in which the correction unit 44 performs the correction on the metal regions in the low-energy projection data and the high-energy projection data, but a form may also be employed in which the correction unit 44 performs the correction on the metal regions in difference data between the low-energy projection data and the high-energy projection data. That is, the correction target data of the present disclosure may be, for example, low-energy projection data and high-energy projection data, or may be difference data between the low-energy projection data and the high-energy projection data.

Additionally, in the above-described embodiment, a form has been described in which the console 30 acquires the projection data of two types of energies, that is, the low energy and the high energy, from the detector 28 of the CT apparatus 10, but a form may also be employed in which projection data of three types of energies are acquired. For example, a form may also be employed in which the detector 28 of the CT apparatus 10 detects three types of projection data, that is, low-energy projection data, medium-energy projection data, and high-energy projection data, each corresponding to emitted radiation of a different energy.

In addition, the correction unit 44 may specify the metal region in the image space based on the amount of change derived based on a difference or a ratio between a low-energy reconstructed image obtained by reconstructing the low-energy projection data and a high-energy reconstructed image obtained by reconstructing the high-energy projection data. For example, in a case of an imaging method in which the low-energy projection data and the high-energy projection data at the same projection angle are not present, such as in a case where low-energy projection data is acquired by emitting radiation of low-energy at a certain projection angle, and high-energy projection data is acquired by emitting radiation of high-energy at the next projection angle while acquiring projection data at various projection angles, a form may be employed in which the correction unit 44 specifies the metal region in the image space, instead of specifying the metal region in the above-mentioned projection space. For example, as a result of performing the correction process including the interpolation process in the projection space, projection data from which a metal component has been removed may be generated depending on the processing. In such a case, it is preferable that the metal region can be specified in the image space in order to add metal information in the image space.

In the above-described embodiment, a form has been described in which the console 30 comprises three processing units, that is, the acquisition unit 40, the specification unit 42, and the correction unit 44, but a form may also be employed in which the functions of these processing units are performed by two or fewer processing units or four or more processing units. For example, a form may be employed in which one processing unit having functions of the specification unit 42 and the correction unit 44 performs the correction process of correcting artifacts in the region (the metal region in the embodiment described above) in which the amount of change between the low-energy projection data and the high-energy projection data is equal to or greater than the threshold value.

In addition, in the above-described embodiment, a form has been described in which the specification unit 42 specifies the metal region, and the correction unit 44 corrects artifacts in the metal region specified by the specification unit 42, but the region to be specified and corrected is not limited to the metal region. The region to be specified and corrected need only be a region in which the amount of change between the low-energy projection data and the high-energy projection data is equal to or greater than the threshold value, and may be, for example, a region of a bone or the like.

In the above-described embodiment, each process is executed by any computer. Additionally, any computer may execute these processes by means of a processor as hardware, a program as software, or a combination thereof. In that case, the processor is configured to execute various types of processes in the present embodiment in cooperation with the program and can function as each unit or each means in the present embodiment. Further, the execution order of the process by the processor is not limited to the order described above and may be changed as appropriate. Any computer may be a general-purpose computer, a computer for a specific use, a workstation, or another system capable of executing each process.

The processor may be configured using one or a plurality of pieces of hardware, and a type of hardware is not limited. For example, the processor may be configured using hardware such as a central processing unit (CPU), a micro processing unit (MPU), a programmable logic device such as a field programmable gate array (FPGA), a dedicated circuit for executing specific processing such as an application-specific integrated circuit (ASIC), a graphic processing unit (GPU), or a neural processing unit (NPU). Additionally, the type of hardware may be a combination of different types of hardware. In a case where a plurality of pieces of hardware are configured to execute one or a plurality of processes of a certain processor, the plurality of pieces of hardware may be present in devices physically separated from each other or may be present in the same device. Further, in any of the embodiments, the order of each process by the processor is not limited to the above-described order and may be changed as appropriate. The hardware is configured using an electrical circuit (circuitry) in which circuit elements such as semiconductor elements are combined, or the like.

Furthermore, the program may be software such as firmware or a microcode. In addition, the program may be, for example, a program module group, and each function thereof may be implemented by a processor configured to execute the corresponding function. The program may be a program code or a plurality of code segments stored in one or a plurality of non-transitory computer-readable media (for example, storage media, other storages, or the like). The program may be stored in a distributed manner across a plurality of non-transitory computer-readable media that are present in devices physically separated from each other. The program code or code segments may represent any combination of procedures, functions, subprograms, routines, subroutines, modules, software packages, classes, or commands, data structures, or program statements. The program code or code segments may be connected to other code segments or hardware circuits by transmitting and receiving information, data, arguments, parameters, or contents of a memory.

Additionally, in the above-described embodiment, an aspect has been described in which the information processing program 33 is stored (installed) in the storage unit 34 of the console 30 in advance, but the present disclosure is not limited to this. The information processing program 33 may be provided in a form recorded on a recording medium such as a compact disc read only memory (CD-ROM), a digital versatile disc read only memory (DVD-ROM), and a universal serial bus (USB) memory. Alternatively, the information processing program 33 may be provided in a form that can be downloaded from an external device via a network.

In addition, the technology of the present disclosure extends to all program products. The program product includes all forms of products for providing a program. For example, the program product includes a program provided through a network such as the Internet, a non-transitory computer-readable recording medium such as a CD-ROM, a DVD, and a USB memory in which the program is stored, and the like.

Additionally, it goes without saying that the configurations, operations, and the like of the CT apparatus 10, the console 30, and the like described in each of the above-described embodiments are merely examples and can be changed depending on the situation within the scope of the present invention without departing from its gist. Further, it goes without saying that the above-described embodiments may be combined as appropriate.

The following supplementary notes are disclosed with respect to the above-described embodiments.

### Supplementary Note 1

An Information Processing Apparatus Comprising:
a processor,
in which the processor is configured to:
   acquire first projection data output from a detector that has detected radiation of a first energy transmitted through a subject and second projection data output from the detector that has detected radiation of a second energy transmitted through the subject, the second energy being different from the first energy; and
   perform a correction process of correcting artifacts in a region in which an amount of change between the first projection data and the second projection data is equal to or greater than a threshold value.

### Supplementary Note 2

The information processing apparatus according to Supplementary Note 1, in which the processor is configured to:
   perform, as the correction process, a process of correcting the region of correction target data by using interpolation data, the correction target data being the first projection data and the second projection data, or difference data between the first projection data and the second projection data; and
   generate an interpolation-processed reconstructed image by reconstructing the corrected correction target data.

### Supplementary Note 3

The information processing apparatus according to Supplementary Note 2, in which the processor is configured to:
   generate an interpolation error reduction image in which an interpolation error component is reduced from the interpolation-processed reconstructed image;
   generate interpolation error reduction forward-projection data obtained by forward-projecting the interpolation error reduction image;
   perform a replacement process on the region of the correction target data based on the interpolation error reduction forward-projection data such that continuity between the region and an adjacent region is increased; and
   perform a residual error reduction process on the correction target data after the replacement to generate corrected projection data.

### Supplementary Note 4

The information processing apparatus according to Supplementary Note 3, in which the processor is configured to:
   perform the replacement process by using any of baseline shift or normalized interpolation.

### Supplementary Note 5

The information processing apparatus according to Supplementary Note 3 or 4, in which the processor is configured to:
   perform the residual error reduction process on error projection data obtained by subtracting a metal component corresponding to metal and the interpolation error reduction forward-projection data from the correction target data.

### Supplementary Note 6

The information processing apparatus according to any one of Supplementary Notes 3 to 5,
in which the processor is configured to:
   perform the residual error reduction process based on frequency information.

### Supplementary Note 7

The information processing apparatus according to Supplementary Note 6, in which the processor is configured to:
   perform, as the residual error reduction process, a weighted addition in which a weight of a frequency component other than a high-frequency component corresponding to noise and a low-frequency component corresponding to artifacts is greater than weights of the high-frequency component and the low-frequency component.

### Supplementary Note 8

The information processing apparatus according to any one of Supplementary Notes 3 to 7,
in which the processor is configured to:
   generate an interpolation error reduction image in which a pixel value of a pixel having a pixel value within a predetermined range is replaced with a pixel value different from the pixel value.

### Supplementary Note 9

The information processing apparatus according to any one of Supplementary Notes 1 to 8,
in which the processor is configured to:
   derive the amount of change based on a difference or a ratio between the first projection data and the second projection data.

### Supplementary Note 10

An information processing method comprising:
causing a processor to execute:
   acquiring first projection data output from a detector that has detected radiation of a first energy transmitted through a subject and second projection data output from the detector that has detected radiation of a second energy transmitted through the subject, the second energy being different from the first energy; and
   performing a correction process of correcting artifacts in a region in which an amount of change between the first projection data and the second projection data is equal to or greater than a threshold value.

### Supplementary Note 11

An information processing program for causing a processor to execute a process comprising:
acquiring first projection data output from a detector that has detected radiation of a first energy transmitted through a subject and second projection data output from the detector that has detected radiation of a second energy transmitted through the subject, the second energy being different from the first energy; and
performing a correction process of correcting artifacts in a region in which an amount of change between the first projection data and the second projection data is equal to or greater than a threshold value.

### Explanation of References

What is claimed is:

1. An information processing apparatus comprising:
a processor,
wherein the processor is configured to:
acquire first projection data output from a detector that has detected radiation of a first energy transmitted through a subject and second projection data output from the detector that has detected radiation of a second energy transmitted through the subject, the second energy being different from the first energy; and
perform a correction process of correcting artifacts in a region in which an amount of change between the first projection data and the second projection data is equal to or greater than a threshold value.

2. The information processing apparatus according to claim 1,
wherein the processor is configured to:
perform, as the correction process, a process of correcting the region of correction target data by using interpolation data, the correction target data being the first projection data and the second projection data, or difference data between the first projection data and the second projection data; and
generate an interpolation-processed reconstructed image by reconstructing the corrected correction target data.

3. The information processing apparatus according to claim 2,
wherein the processor is configured to:
generate an interpolation error reduction image in which an interpolation error component is reduced from the interpolation-processed reconstructed image;
generate interpolation error reduction forward-projection data obtained by forward-projecting the interpolation error reduction image;
perform a replacement process on the region of the correction target data based on the interpolation error reduction forward-projection data such that continuity between the region and an adjacent region is increased; and
perform a residual error reduction process on the correction target data after the replacement to generate corrected projection data.

4. The information processing apparatus according to claim 3,
wherein the processor is configured to:
perform the replacement process by using any of baseline shift or normalized interpolation.

5. The information processing apparatus according to claim 3,
wherein the processor is configured to:
perform the residual error reduction process on error projection data obtained by subtracting a metal component corresponding to metal and the interpolation error reduction forward-projection data from the correction target data.

6. The information processing apparatus according to claim 3,
wherein the processor is configured to:
perform the residual error reduction process based on frequency information.

7. The information processing apparatus according to claim 6,
wherein the processor is configured to:
perform, as the residual error reduction process, a weighted addition in which a weight of a frequency component other than a high-frequency component corresponding to noise and a low-frequency component corresponding to artifacts is greater than weights of the high-frequency component and the low-frequency component.

8. The information processing apparatus according to claim 3,
wherein the processor is configured to:
generate an interpolation error reduction image in which a pixel value of a pixel having a pixel value within a specific range is replaced with a pixel value different from the pixel value.

9. The information processing apparatus according to claim 1,
wherein the processor is configured to:
derive the amount of change based on a difference or a ratio between the first projection data and the second projection data.

10. An information processing method comprising:
causing a processor to execute:
acquiring first projection data output from a detector that has detected radiation of a first energy transmitted through a subject and second projection data output from the detector that has detected radiation of a second energy transmitted through the subject, the second energy being different from the first energy; and
performing a correction process of correcting artifacts in a region in which an amount of change between the first projection data and the second projection data is equal to or greater than a threshold value.

11. A non-transitory computer-readable storage medium storing an information processing program for causing a processor to execute a process comprising:
acquiring first projection data output from a detector that has detected radiation of a first energy transmitted through a subject and second projection data output from the detector that has detected radiation of a second energy transmitted through the subject, the second energy being different from the first energy; and
performing a correction process of correcting artifacts in a region in which an amount of change between the first projection data and the second projection data is equal to or greater than a threshold value.
